(19) United States
(12) Patent Application Publication
(10) Pub. No.: US 2008/0108900 A1
(43) Pub. Date: May 8, 2008

(54) Ultrasound transducer apparatus

(76) Inventors: Chih-Kung Lee, Taipei City (TW); Wen-Jong Wu, Chun-Ho City (TW); Chuin-Shan Chen, Taipei (TW); Jay Shieh, Taipei (TW); Wen-Hsin Hsiao, Taiwan (TW); Chih-Chiang Cheng, Taipei (TW); Nien-Ti Tsou, Taipei City (TW); Yao-Tien Huang, Taipei City (TW); Yuan-Ping Liu, Keelung City (TW); Yu-Yuan Chen, Keelung City (TW); Yen-Chieh Wang, Jhong City (TW)

Correspondence Address: ARENT FOX LLP, 1050 CONNECTICUT AVENUE, N.W., SUITE 400, WASHINGTON, DC, 20036, US

(21) Appl. No.: 11/905,158

(22) Filed: Sep. 27, 2007

Related U.S. Application Data
(60) Provisional application No. 60/847,936, filed on Sep. 29, 2006.

(51) Int. Cl. A61B 8/00

(52) U.S. Cl. 600/459

(57) ABSTRACT

An ultrasound transducer apparatus for shaping the ultrasound coverage field per application requirement in both the radiating and receiving mode of operation. Wave coverage field is shaped by forming at least one cut at designated location on the housing wall. A cut on the housing wall of the transducer pot-shaped structural body results into the reduction of wave intensity, either radiating or receiving, toward that direction where the cut is located.

### CROSS-REFERENCE TO RELATED APPLICATIONS

This application is related to the Applicant's co-pending Provisional Application No. 60/847,936 filed Sep. 29, 2006. The entirety of such Provisional Application is incorporated herein by reference.

### FIELD OF THE INVENTION

The present invention relates in general to an ultrasound transducer apparatus. More particularly, this invention relates to an ultrasound transducer apparatus for tailored ultrasound transmission coverage field.

### DESCRIPTION OF THE RELATED ART

Ultrasound transducers are useful in object detection for various applications. Typical applications include target range finding for cameras. Obstacle detection and monitoring at rear proximity of vehicles using ultrasound waves, for example, are also popular. In many cases a piezoelectric element is used for an ultrasound transducer to produce ultrasound waves for these purposes. In most of the cases, an ultrasound transducer is used both as the ultrasound transmitting and receiving means. In other words, the ultrasound transducer is responsible both for generating ultrasound waves used as the scanning and/or ranging wave and for receiving the reflected waves.

Regardless of either the transmitting or the receiving mode an ultrasound transducer is in, the emanating and receiving wave field is often critical to the application. For example, in the automobile rear proximity monitoring application, both the transmitting and receiving ultrasound wave coverage fields are shaped for optimized operation. In general, both coverage fields need to be wide in the horizontal orientation and narrow in vertical. A wide horizontal transmitting coverage field increases the effective angular monitoring range to avoid oversight of objects to the rear of a reversing vehicle. On the other hand, a narrow vertical scanning angular range reduces the likelihood of interference from ultrasound waves reflected from the ground.

Coverage field shaping for the receiving mode of an ultrasound transducer in a vehicle rear proximity monitoring system is basically the same as for the transmitting mode. Similar requirements are substantially applicable to the camera range-finding applications.

Various means for achieving this shaped coverage field in ultrasound transducer applications have been attempted. As is known to those skilled in this art, basic acoustic physics dictates that for an ultrasound transducer with a pot-shaped sound cone there exists a specific inner housing wall configuration that is optimized for shaping the device coverage into a horizontally wide and vertically narrow field. For such an optimized transducer structural configuration, the cross section of the inner housing wall taken along a plane perpendicular to the direction of ultrasound wave radiation resembles that of an upright rectangle or ellipse. Essentially, the contour of the inner wall cross section has a horizontally smaller dimension than the vertical. In other words, an ultrasound transducer having a sound cone with a narrower horizontal dimension and a wider vertical dimension is optimized for generating a coverage field that is wider horizontally than vertically.

In U.S. Pat. No. 6,370,086 “Ultrasound Sensor for Distance Measurement,” Li proposed an ultrasound sensor having a housing for its piezoelectric ultrasound generating element with a unique-shaped chamber. Li attempted to shape up the coverage field of his sensor by modification to the basic vertical rectangular inner opening into several unique shapes, which Li announced to be able to shape the coverage field to a horizontal angular range sufficient to encompass the entire cross section of a vehicle. Li's sensor, however, is not considered far beyond the basic acoustics of the fundamental ultrasonic transducer device for similar applications.

U.S. Pat. No. 6,250,162 “Ultrasonic Sensor” to Amaike et al. disclosed an ultrasonic sensor to shape up the horizontally wide and vertically narrow wave coverage field via a different approach than Li's described above. Amaike et al. used a structured thick-thin configuration over the bottom portion of their sensor. Basic structural configuration of the ultrasonic sensor proposed by Amaike et al., however, is no deviation from the basic one with a housing inner opening that is vertically wider and horizontally narrower.

On the other hand, in a different approach Rapps et al. in U.S. Pat. No. 5,446,332, “Ultrasonic Transducer,” employed damping means attached to the oscillating diaphragm and/or the housing as well as the multiple disposition of piezo oscillators in the system to shape the desired coverage field that is wide horizontally and narrow vertically.

While conventional ultrasound transducers such as those described above are wildly used in applications including vehicle rear proximity monitoring systems, there are still the problems such as insufficient horizontal angular coverage within the range of reasonable detection sensitivity supported by the accompanying electronics. Insufficient horizontal coverage of an ultrasound transducer for a vehicle rear proximity monitoring system implies the need for more transducers so as to cover the entire rear end of a commercial vehicle such as a sedan or a pickup.

Another problem to the opposite is the insufficient narrowness in the vertical direction. Although an old issue, sensitivity of a vehicle rear proximity monitoring system in the vertical direction that is wide enough to cause frequent false detection results into user inconvenience, although not safety related inconvenience.

### SUMMARY OF THE INVENTION

The present invention therefore provides an ultrasound transducer able to provide improved coverage characteristics for meeting specific application requirements. Wave coverage field featured by an ultrasound transducer of the present invention is shaped by the provision of at least one cut at designated location on the housing wall. A cut on the housing wall of the transducer pot-shaped structural body results into the reduction of wave intensity, either radiating or receiving, toward that direction where the cut is located.

The present invention further provides an ultrasound transducer that provides improved coverage characteristics. Shaping of the wave coverage is by forming at least one thickness reduction area at designated location on the housing wall. A thickness reduction area on the housing wall of the transducer pot-shaped structural body results into the reduction of wave intensity, either radiating or receiving, toward that direction where the cut or thickness reduction area is located. A recess formed as a result of the presence of a thickness reduction area may be a recess on the inner or outer surface of the housing wall.

### BRIEF DESCRIPTION OF THE DRAWING

FIGS. 1A and 1B respectively are views from different perspective of a prior art transducer for ultrasound proximity monitoring system.

FIG. 2 illustrates in perspective another prior art transducer for ultrasound proximity monitoring systems.

FIG. 3 illustrates in perspective yet another prior art transducer for ultrasound proximity monitoring systems.

FIG. 4A is a perspective view of a piezoelectric transducer in accordance with a preferred embodiment of the present invention.

FIG. 4B is a side elevational view of the transducer of FIG. 4A.

FIG. 5 is a perspective view of a piezoelectric transducer in accordance with another embodiment of the present invention.

FIG. 6 is a perspective view of a piezoelectric transducer in accordance with yet another embodiment of the present invention.

FIG. 7 is a perspective view of a piezoelectric transducer in accordance with still another embodiment of the present invention.

FIGS. 8A and 8B are perspective views of still yet another embodiment of the present invention.

FIG. 8C is a side elevational view of the transducer of FIGS. 8A and 8B.

FIG. 9 shows the wave intensity characteristic of a conventional ultrasound transducer for commercial vehicle rear proximity monitoring system.

FIG. 10 shows the wave intensity characteristics of a transducer made in accordance with the present invention using the transducer tested in FIG. 9 as the basic construction element.

### DETAILED DESCRIPTION OF THE PREFERRED EMBODIMENT

An ultrasound transducer apparatus in accordance with the present invention provides improved coverage characteristics to meet specific application requirements. The ultrasound transducer apparatus of the present invention is capable of providing coverage of specific angular range along any desired directional span, either horizontal, vertical or any direction in between per the application requires. The ultrasound transducer apparatus of the present invention is able to achieve this in both the wave radiating and detecting modes of operation.

The present invention achieves shaping of the ultrasound wave coverage field by an inventive transducer apparatus having at least one cut or thickness reduction area at designated location on the housing wall. The inventive characteristics housing wall cut or cuts are penetrating cuts, meaning they cut through the entire wall thickness of the transducer ultrasound cone over an area with selected shape. On the other hand, a housing wall thickness reduction area for the transducer apparatus of the present invention is an area of designated shape and size over the surface of the housing wall that has a smaller thickness than the surrounding area of the housing wall.

Essentially, location of a penetrating cut or a thickness reduction site on the transducer housing wall is functionally related to the wave field shaping characteristics of the transducer apparatus of the present invention, as will be described in detail in the following paragraphs via description to various preferred embodiments of the present invention.

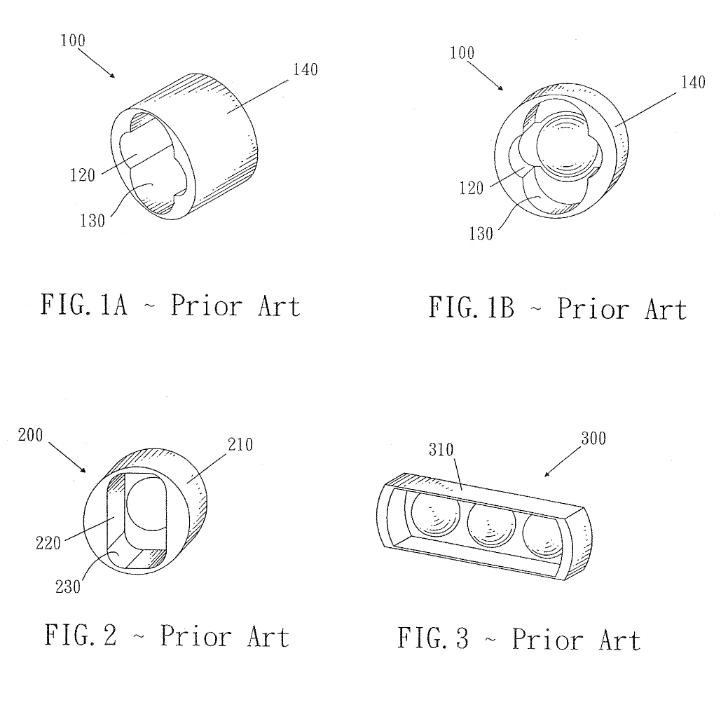

FIGS. 1A and 1B respectively are views from different perspective of a prior art ultrasound transducer typically used for vehicle rear proximity monitoring systems. The transducer of FIGS. 1A and 1B is closely similar to a commercial transducer manufactured such as by Murata Manufacturing Company, Ltd. of 10-1, Higashikotari 1-chome, Nagaokakyo-shi, Kyoto 617-8555, Japan. FIG. 1A illustrates the generally cylindrical pot-shaped transducer housing 110 of the prior art ultrasound transducer 100. Pot opening 120 opened toward the front lower left of the transducer 100 as viewed into the drawing reveals a generally elongated and upright cross-sectional contour of the inner housing wall 130. This upright elongated cross-sectional contour is better shown in the perspective view of FIG. 1B as is signified by the fact that the housing wall at the top and bottom peripheral locations is thinner than that at the left and right peripheral locations.

As described above, although Amaike in U.S. Pat. No. 6,250,162 used a structured thick-thin configuration over the bottom portion for his transducer, basic structural configuration of the entire device of FIGS. 1A and 1B is substantially in compliance with a housing inner opening contour that is vertically wider and horizontally narrower. This allows the transducer 100 of FIGS. 1A and 1B to shape up an ultrasound wave coverage field that is wide horizontally and narrow vertically, a characteristic suitable for vehicle rear proximity monitoring systems.

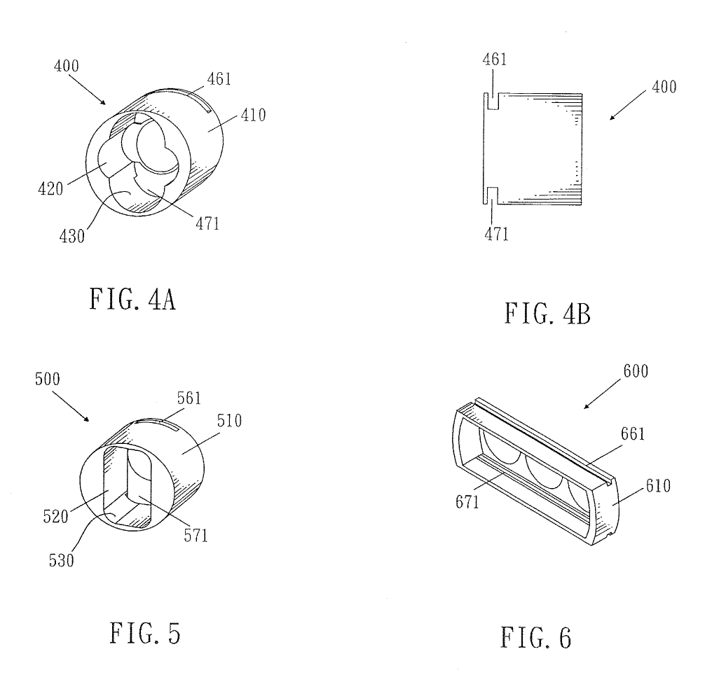

FIG. 4A is a perspective view of a piezoelectric transducer in accordance with a preferred embodiment of the present invention and FIG. 4B is a side elevational view of the same transducer. The inventive transducer apparatus 400 of FIG. 4A can be considered to be an implementation of the fundamental idea of the present invention utilizing the prior art device 100 of FIGS. 1A and 1B as the basic construction element.

Based on the basic structure of the transducer 100, two cuts 461 and 471 are formed on the cylindrical wall of the pot-shaped housing 410. As is comprehensible for those in the art, these cuts 461 and 471 can be formed by machining to the housing structure of a conventional device, transducer 100 in this exemplified case, or, the cuts may be formed along with the formation of the housing 410 for the device.

In this described embodiment of FIG. 4A, cuts 461 and 471 are elongated slots formed along the peripheral circumference of the housing 410. Length of each cut can also be set with flexibility to cope with the targeted wave field shaping requirement. In this illustrated example, cuts 461 and 471 are shown each to be roughly occupying 90 degrees of the entire periphery of the housing.

Moreover, in this described example, cuts 461 and 471 are opposite to each other with respect to the center of the device 400. Further, cuts 461 and 471 of the device 400 are also shown to be formed at an axial location on the housing 410 that is close to the bottom surface of the device 400. As is well known in this art, this bottom surface is where the ultrasound generating means, normally a piezoelectric element, is located. All these structural features of the cuts with respect to the basic device housing as described are better observed if reference is made simultaneously to the side elevational view of FIG. 4B.

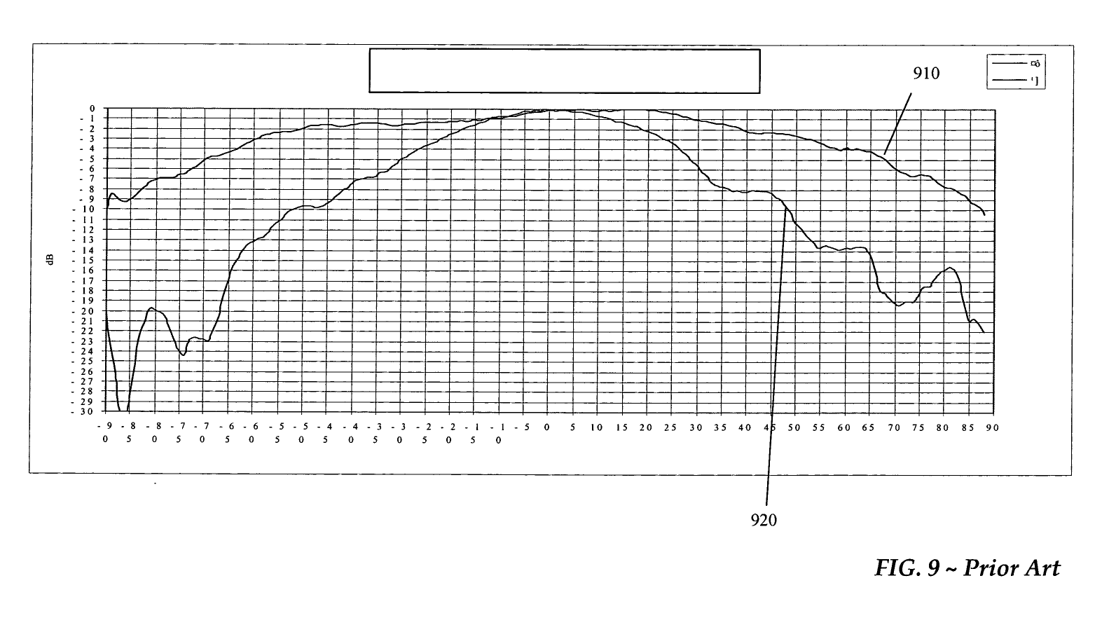

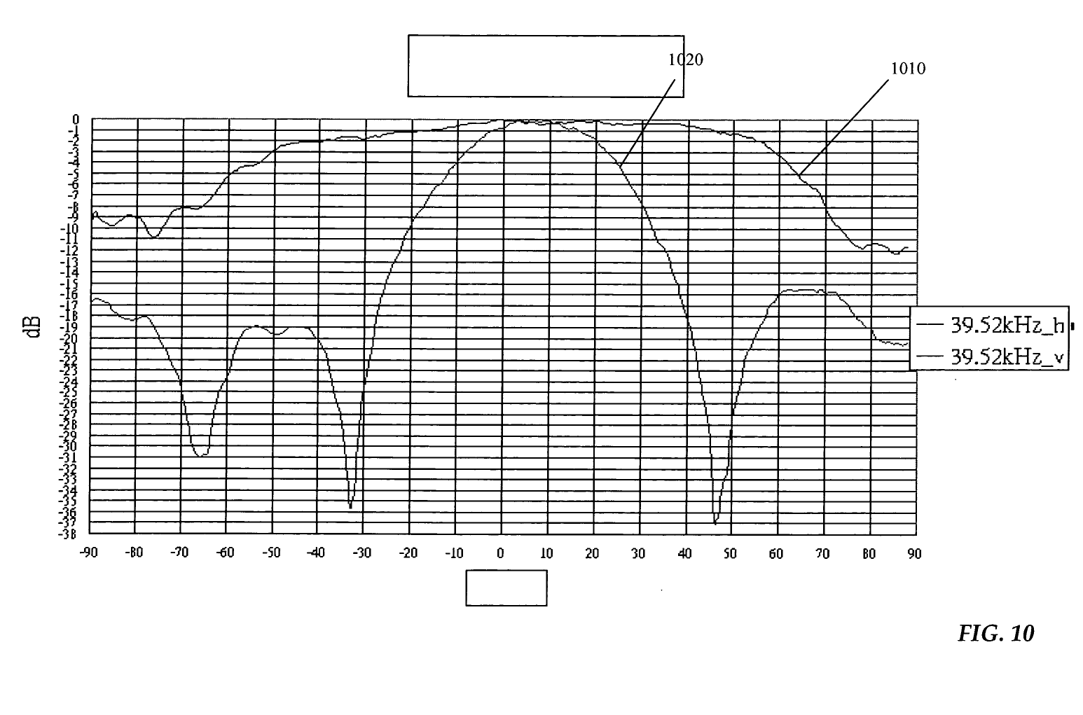

Cuts 461 and 471 formed on the side wall of housing 410 of the inventive device 400 provide improved control over the effort of shaping the ultrasound wave field for meeting specific application requirement. Experimental results outlined in FIGS. 9 and 10 clearly evidence this. FIG. 9 is the wave intensity characteristic diagram of a conventional ultrasound transducer for use in commercial vehicle rear proximity monitoring system, and FIG. 10 shows the characteristics under the same testing conditions for a transducer made in accordance with the present invention using the same transducer tested in FIG. 9 as the basic construction element.

Ultrasound radiation coverage field of the tested transducer as shown in FIG. 9 was measured at the frequency of 40 kHz, typical of many commercial car rear proximity monitoring systems. The tested device was commercially designed, made and marketed for this specific application, as is revealed by the measured characteristics: The horizontal coverage characteristics indicated by curve 910 shows that at the cut-off intensity of −3 dB, the tested device covers a horizontal angular range of about 110 degrees, from −59 degrees at left to +52 right. The vertical coverage at the same −3 dB level was about 50 degrees, from down −23 degrees to up +25 as the characteristic curve 920 indicates.

After machining was applied to the same test transducer that forms one cut at the lower peripheral circumference of the pot-shaped housing substantially the same as that shown as cut 471 in the exemplified device of FIGS. 4A and 4B, the device became an experimental specimen embodying the idea of the present invention. The inventive transducer was then tested under the same setup and under the same test frequency as that used to obtain the pre-machining test result of FIG. 9.

An examination to the test result of FIG. 10 reveals the fact that the horizontal coverage characteristics, represented by curve 1010, of the machined transducer was not altered, still covers about 110 degrees, horizontally from left −51 degrees to right +59 at −3 dB. However, as a result of the cut formed on the bottom side of the transducer, the vertical coverage characteristics had been changed. The vertical coverage range represented by curve 1020 was about 30 degrees, from up +22 degrees to down −8 at −3 dB.

Note that the upward coverage angle was +22 degrees above the horizontal reference plane, considered the same as the +24 degrees of the pre-machined device within experimental tolerances. However, due to the presence of the cut at the bottom side of the device, the downward coverage angle (with respect to the horizontal reference plane) become reduced to −8 degrees.

This adds up to about 30 degrees vertical coverage, almost 20 degrees improvement with respect to the 50 degrees vertical coverage achieved by the device tested in FIG. 9. Such an improvement is significant for commercial vehicle rear proximity monitoring applications. Reduced vertical coverage range reduces the probability of false detection due to the presence of nearby objects close to the rear of the vehicle but poses no substantial safety hazards.

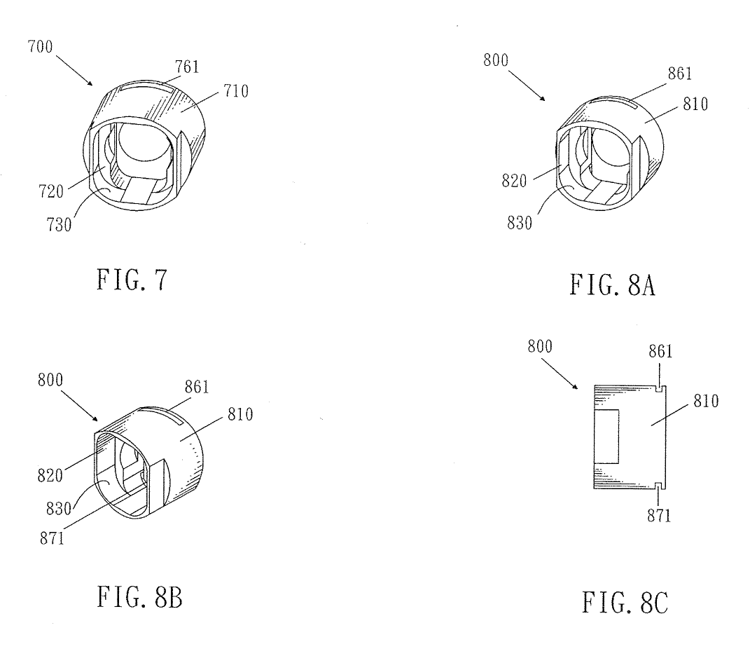

As described above, the inventive ultrasound transducer apparatus tested to result into the coverage characteristics of FIG. 10 had only one bottom side cut. FIG. 7 shows another embodiment of the one-cut implementation of the idea of the present invention. As is shown, the embodiment of FIG. 7 is an ultrasound transducer 700 featuring a top side cut 761. Such a one-cut transducer features a wave coverage characteristics that is with reduced top coverage angle when compared to one without any cut.

Per the application requirement calls for, the transducer may also be equipped with a top side cut so that the coverage range in the vertical direction above the horizontal reference plane can also be reduced. This will require a two-cut device, with one cut at the top side and another at the opposite bottom side. In addition to the depicted embodiment of FIGS. 4A and 4B, the transducer 800 illustrated in FIGS. 8A, 8B and 8C represents another implementation of the present invention. Transducer 800 has the basic housing construction the same as transducer 700 of FIG. 7. The exemplified transducer 700 has a substantially pot-shaped housing 710, and with an opening 720 of round-corners and upright rectangular inner wall contour in cross section.

FIG. 2 illustrates in perspective another prior art transducer for ultrasound proximity monitoring systems, and FIG. 5 is a perspective view of a piezoelectric transducer 500 in accordance with another embodiment of the present invention. Transducer 500 can be considered to be one made using the prior art transducer 200 of FIG. 2. Similar to the transducer 400 of FIGS. 4A and 4B, the transducer embodiment 500 of FIG. 5 also features a pot-shaped housing 510, but with an opening 520 having a generally round-cornered, upright rectangular inner wall contour in cross section. The transducer 500 has two housing wall cuts 561 and 571 located opposite to each other at the top and bottom side of the housing 510. This two-cut transducer 500 therefore has a vertical coverage further narrower than that featured by transducer 700 of FIG. 7.

FIG. 3 illustrates in perspective yet another prior art transducer for ultrasound proximity monitoring systems. By contrast, the perspective view of FIG. 6 shows yet another transducer of according to the present invention. Ultrasound transducer 600 has two cuts 661 and 671 formed on the top and bottom sides of the housing 610. It is made using a transducer with multiple disposition piezo oscillators substantially the same as that disclosed in U.S. Pat. No. 5,446,332 discussed above as the basic structural element. Essentially the embodiment transducer 600 is made by forming the top and bottom cuts 661 and 671 into the housing of the prior art transducer 300 shown in FIG. 3. Vertical coverage of the inventive transducer 600 of FIG. 6 therefore is relatively reduced to a narrower angle with respect to the prior art transducer 300 of FIG. 3.

Thus, an ultrasound transducer apparatus in accordance with the present invention is able to provide improved coverage characteristics for meeting specific application requirements. Shaping of the wave coverage field as featured by an ultrasound transducer of the present invention is possible by forming at least one cut or thickness reduction area at designated location on the housing wall. Essentially a cut or a thickness reduction area on the housing wall of the transducer pot-shaped structural body results into the reduction of wave intensity, either radiating or receiving, toward that direction where the cut or thickness reduction area is located. Therefore, housing wall cuts or recessed areas formed by the presence of thickness reduction areas become means for adjusting shaping of the wave coverage field for that transducer. Note that a recess formed as a result of the presence of a thickness reduction area refers to a recess area over the inner surface of the housing wall.

While the above is a full description of the specific embodiments, various modifications, alternative constructions and equivalents may be used. Therefore, the above description and illustrations should not be taken as limiting the scope of the present invention, which is defined by the appended claims.

What is claimed is:

1. An ultrasound transducer apparatus having a pot-shaped cylindrical housing for radiating ultrasound waves out of a pot opening of said housing and for receiving ultrasound waves into said pot opening, said apparatus comprising:
at least one cut on said housing for reducing radiated and received wave intensity in the coverage field of said apparatus toward where said at least one cut is located.

2. The ultrasound transducer apparatus of claim 1, wherein said at least one cut forms an opening penetrating through the thickness of said housing.

3. The ultrasound transducer apparatus of claim 1, wherein said at least one cut forms a thickness-reduced area of said housing.

4. The ultrasound transducer apparatus of claim 3, wherein said thickness-reduced area is a recessed area over the external surface of said housing.

5. The ultrasound transducer apparatus of claim 3, wherein said thickness-reduced area is a recessed area over the internal surface of said housing.

6. The ultrasound transducer apparatus of claim 3, wherein said thickness-reduced area is a recessed area over both the external and internal surface of said housing.

7. The ultrasound transducer apparatus of claim 1, wherein said at least one cut is an elongated slot having a length extending approximately 90 degrees of entire periphery of said pot-shaped cylindrical housing.

8. An ultrasound transducer apparatus having a pot-shaped cylindrical housing for radiating ultrasound waves out of a pot opening of said housing and for receiving ultrasound waves into said pot opening, said apparatus comprising:
two cuts located opposite to each other on said housing for reducing radiated and received wave intensity in the coverage field of said apparatus toward where said two cuts are located.

9. An ultrasound apparatus comprising:
a transducer having at least one cut on the transducer housing for reducing radiated and received wave intensity in the coverage field of said transducer toward where said at least one cut is located.

10. An ultrasound transducer comprising:
at least one cut on the housing thereof for reducing radiated and received wave intensity in the coverage field of said transducer toward where said at least one cut is located.

11. A vehicle rear proximity monitoring system comprising:
an ultrasound transducer having a pot-shaped cylindrical housing for radiating ultrasound waves out of a pot opening of said housing and for receiving ultrasound waves into said pot opening, said apparatus comprising
at least one cut on said housing for reducing radiated and received wave intensity in the coverage field of said apparatus toward
where said at least one cut is located.

12. The vehicle rear proximity monitoring system of claim 11, wherein said at least one cut is toward ground.

13. A vehicle rear proximity monitoring system comprising:
an ultrasound transducer having a pot-shaped cylindrical housing for radiating ultrasound waves out of a pot opening of said housing and for receiving ultrasound waves into said pot opening, said apparatus comprising
two cuts located opposite to each other on said housing for reducing radiated and received wave intensity in the coverage field of said apparatus toward where said two cuts are located.

14. The vehicle rear proximity monitoring system of claim 13, wherein said at least one cut is toward ground.
